(19) United States
(12) Patent Application Publication
(10) Pub. No.: US 2023/0097267 A1
(43) Pub. Date: Mar. 30, 2023

(54) COMPUTER-IMPLEMENTED METHOD FOR EVALUATING AN IMAGE DATA SET OF AN IMAGED REGION, EVALUATION DEVICE, IMAGING DEVICE, COMPUTER PROGRAM AND ELECTRONICALLY READABLE STORAGE MEDIUM

(71) Applicant: Siemens Healthcare GmbH, Erlangen (DE)

(72) Inventors: Chris SCHWEMMER, Forchheim (DE); Thomas ALLMENDINGER, Forchheim (DE); Rainer GRIMMER, Erlangen (DE)

(73) Assignee: Siemens Healthcare GmbH, Erlangen (DE)

(21) Appl. No.: 17/948,573

(22) Filed: Sep. 20, 2022

(30) Foreign Application Priority Data
Sep. 24, 2021 (EP) 21198958.7

(51) Int. Cl. G06T 7/00; G06T 7/11; G06T 7/174; A61B 6/00

(52) U.S. Cl. CPC G06T 7/0014; G06T 7/11; G06T 7/174; A61B 6/5217; G06T 2207/10081; G06T 2207/30104; G06T 2207/20021

(57) ABSTRACT

A computer-implemented method for evaluating an image data set of an imaged region comprises: determining, from the image data set, at least two processed data sets having different image data content; applying a first sub-algorithm, of an evaluation algorithm, to a first of at least two processed data sets to determine a first intermediate result relating to image data content of the first of the at least two processed data sets; applying a second sub-algorithm, of the evaluation algorithm, to a second of the at least two processed data sets to determine a second intermediate result relating to image data content of the second of the at least two processed data sets; determining quantitative evaluation result data by a third sub-algorithm of the evaluation algorithm, wherein the third sub-algorithm uses both the first intermediate result and the second intermediate result as input data.

### CROSS-REFERENCE TO RELATED APPLICATION(S)

The present application claims priority under 35 U.S.C. § 119 to European Patent Application No. 21198958.7, filed Sep. 24, 2021, the entire contents of which are incorporated herein by reference.

### FIELD

One or more example embodiments of the present invention concern a computer-implemented method for evaluating an image data set of an imaged region, in particular of a patient, wherein, from the imaged data set, different processed data sets having different image data content are determinable by image processing. One or more example embodiments of the present invention further concern an evaluation device, an imaging device, a computer program and an electronically readable storage medium.

### BACKGROUND

Advanced imaging techniques, in particular regarding computed tomography and magnetic resonance, provide an increasing amount of information regarding an imaged region, in particular regarding medical imaging, which often is a foundation of diagnostics. However, this information often cannot be directly seen from the acquired image data set, such that evaluation algorithms applied for evaluation of the image data set become more important, in particular in clinical routine. Some of these evaluation algorithms also employ artificial intelligence, in particular by comprising trained functions, for example neural networks. Evaluation algorithms, due to complexity, are usually developed to use one data set as input data, in particular the, for example pre-processed, image data set itself. For example, trained functions may be trained on a certain type of data set as input data.

Since, traditionally, evaluation algorithms, in particular for medical image data, work on single data sets, for example one reconstructed computed tomography volume with specific acquisition and reconstruction parameters, the information that the evaluation algorithm should extract should be visible in the single input data set. For example, an algorithm for detecting landmarks is required to run on data where these landmarks are visible, for example data acquired using a contrast agent. However, if an evaluation question requires different data, for example without contrast agent or functional image data that does not show any anatomy, such a landmark detection algorithm could not be used. Hence, evaluation is limited to the image information available in a certain data set. If several image data sets from different acquisitions were to be used, they would have to be registered, since those image data sets would not be aligned. On the other hand, from a clinical perspective, often both a contrasted and a non-contrasted scan are acquired, but it would be desirable to skip one scan to reduce the x-ray dose for the patient.

If only a single data set, for example image data set or processed data set, is used for an evaluation algorithm, the possibilities are reduced. For example, an automatic coronary calcium scoring requires non-contrasted input data to reliably identify classifications in the image data set. To assign these classifications to certain blood vessels, a respective segmentation would be highly beneficial, which is, however, strongly limited on non-contrasted data. Hence, the accuracy of such an assignment is also limited. Comparable problems arise in automatic heart valve calcium scoring, wherein, additionally, the heart valves are not aligned with axial images derivable from the image data set, while a valve-oriented view would be highly desirable for reading. Finding such a view is difficult on non-contrasted data, which is an additional problem to assigning classifications to valve leaflets.

Regarding the problem of spine and rib unfolding, non-contrasted data is required to reliably identify the spinal channel. On a contrasted acquisition, differentiation between the contrasted blood vessels and the spinal channel would be very difficult. In some cases, evaluations are not even possible using a single data set, for example for automatic anatomy-based image reformations for non-anatomical image data.

To solve this problem, it was proposed to acquire two image data sets and perform a registration. However, workflow and registration quality issues arise. For example, in automatic coronary and/or valve calcium scoring, the non-contrasted scan is usually performed before the contrasted scan, so that the latter is not available at the same time. In addition, due to the movement of the heart and the patient and the difficulty of finding anatomical landmarks in the non-contrasted data, registration is difficult and may lead to imperfect results, in particular artifacts. In another example, namely automatic anatomy-based image reformations for non-anatomical image data, due to the movement of the target anatomy and the patient and the difficulty of finding anatomical landmarks in non-anatomical image data, registration is difficult and may also lead to artifacts. Finally, regarding the automatic determination of the extra-cellular volume (ECV) or late enhancement in the myocardium, a segmentation of the blood pool and the myocardial segments based on a prior contrasted scan is required in addition to an extra non-contrast scan taken before for determination of the baseline.

### SUMMARY

It is an object of one or more example embodiments of the present invention to provide improved evaluation quality and improved reliability of quantitative evaluation results.

At least this object is achieved by providing a computer-implemented method, an evaluation device, an imaging device, a computer program and an electronically readable storage medium according to one or more example embodiments of the present invention.

In a computer-implemented method for evaluating an image data set of an imaged region, according to one or more example embodiments of the present invention, an image data set from which different processed data sets having different image data content are determinable by image processing is used. To determine quantitative evaluation result data describing at least one dynamic and/or static feature of the imaged region by applying an evaluation algorithm, the method comprises

determining at least two processed data sets having different image data content from the image data set,

applying a first sub-algorithm of the evaluation algorithm to a first of the processed data sets to determine a first intermediate result relating to the image data content of the first processed data set,

applying a second sub-algorithm of the evaluation algorithm to a second of the processed data sets to determine a second intermediate result relating to the image data content of the second processed data set, and

determining the quantitative evaluation data by a third sub-algorithm of the evaluation algorithm, wherein the third sub-algorithm uses both the first and the second intermediate results as input data.

Quantitative evaluation result data may, for example, comprise physical quantities not directly obvious/visible from the image data and/or non-localized physical quantities and/or physical quantities relating to semantical entities of the imaged region, for example certain anatomical features of a patient. In particular, the method is applied in medical imaging, such that the imaged region may be or comprise a region of interest of the patient. The image data set is preferably three-dimensional. For example, the image data set may be a magnetic resonance data set or a computed tomography data set. While most examples discussed here will be related to medical imaging, it is noted that the principles described herein may also be applied in other applications, for example material testing.

From the image data set, different processed data sets having different image data content may be determinable. Different image data content means that different properties of the imaged region are visible in or derivable from the respective processed data sets. Advanced imaged processing techniques allow a plurality of processing variants to emphasize certain image contents/properties of the image region or, in particular, even allow their visualization. Besides using different filter kernels in computed tomography to create clearly different image impressions, a large plurality of processed data sets is conceivable using spectral computed tomography, which may also be called multi energy computed tomography or generally multi energy imaging.

That is, preferably, the image data set may be a multi energy computed tomography data set. In multi energy computed tomography, by applying corresponding image processing, image properties may be fundamentally changed, such that comparison/similarity of the resulting processed data sets may not even be provided. In particular, monoenergetic images may be determined as processed data sets, while, preferably, at least one of the processed data sets may be determined based on a material decomposition. In a material decomposition, different materials in the imaged region can be extracted or differently emphasized or suppressed.

In other words, using spectral computed tomography image data sets, it is possible to reconstruct very different image impressions from the same acquired image data set. For example, certain materials may be suppressed completely or their representation may be enhanced.

In particular, the imaged data set may be acquired using source-based and/or detector-based multi energy computed tomography, in particular using a counting x-ray detector. Source-based methods include, for example, dual layer computed tomography and kV switching technologies. However, since in source-based technologies, time differences may occur, even if they are small, detector-based computed tomography is preferred. Here, the processed data sets will be perfectly registered to each other since they have been acquired at the same time. In particularly preferred embodiments, a counting x-ray detector is used, which allows to separately measure single events and their x-ray photon energy, so that, for example, each single event may be sorted into one of multiple energy slots.

If the multi energy computed tomography image data set is an angiography data set, at least one of the processed data sets may be chosen from the group comprising a virtual non-contrast image, an iodine concentration image, a functional, in particular perfusion, image, a monoenergetic image, a virtual non-calcium image, and a virtual non-iodine image. For angiography, usually, an iodine contrast agent is used. However, techniques for image processing comprise determining a virtual non-contrast image from a iodine contrasted scan, which mimics a native non-contrast scan. Furthermore, iodine concentration images providing a quantitative iodine map of the individual voxels may also be determined. Functional images, for example regarding perfusion of tissue, may be derived from late enhancement contrasted image data sets. Additionally, monoenergetic images, in particular kV images at different kV level settings may be calculated. For example, very high kV images enable a view into metallic objects and low kV settings provide images with high anatomical detail. In a virtual non-calcium reconstruction, a non-calcium or calcium-subtracted image representation is provided. A virtual non-iodine image is similar to a virtual non-contrast image, however, the Hounsfield unit (HU) values of calcium or bone structures are preserved, such that such images may, for example, be used for calcium scoring.

While many examples discussed here may refer to coronary angiography, of course, other applications and medical imaging are also conceivable. For example, regarding liver tumors, contrasted computed tomography scans may also be performed to, for example, assess the blood perfusion of the tumor. This is particularly important regarding therapy monitoring, where, for example, as quantitative evaluation data, relative perfusion of the tumor and the liver may be determined.

Generally, in one or more example embodiments of the present invention, the use of at least two processed data sets having different image data content and the combination of respective evaluation results leads to an improved quality of the final results, in particular by combining anatomical information with other, for example functional, information. In particular when using multi energy computed tomography, no registration between different representations of the imaged region is required, such that faster processing, less artifacts and in particular perfect spatial match is achieved. In particular, a separate scan in computer tomography can be avoided by generating virtual non-contrast data sets out of a contrasted acquisition. Generally, an enhanced amount of information is provided to the evaluation algorithm. Using multiple representations of the imaged region, that is multiple processed data sets, allows the determination of additional information which cannot be derived from only a single data set.

It is noted that, while two processed data sets are explicitly discussed as an example, it is also possible to use more than two processed data sets providing further intermediate results in addition to the first and second intermediate results, which may also be used by the subsequent third sub-algorithm as input data. In other embodiments, cascaded evaluations may also be possible, for example by combining at least one pair of intermediate results into another intermediate results, which may then be used as input data for a subsequent sub-algorithm. In other words, it can be generally said that one or more first sub-algorithms, second sub-algorithms and third sub-algorithms may be applied to one or more processed data sets, second processed data sets, or one or more first and second intermediate results, respectively.

In preferred embodiments, the first intermediate result is or describes a segmentation result regarding multiple segmented features, wherein the third sub-algorithm assigns data of the second intermediate result to segmented features to yield quantitative segmented feature-specific evaluation results. Regarding medical imaging, for example, the first intermediate result may describe anatomy of interest, for example blood vessels in a blood vessel tree of a patient. The second intermediate result may, for example, relate to a quantitative concentration of material, for example calcium, such that it can be determined how much of the material is present in different segmented features, such that quantitative evaluation result data may be provided as amounts or concentrations of material in certain segmented features. Of course, also other quantitative second intermediate results may be assigned to segmented entities, for example perfusion values to certain tissue segments.

As already indicated, the segmented features may preferably be anatomical features and comprise vessels or vessel segments of a vessel tree and/or organs or organ segments. In a concrete embodiment, in multi energy computed tomography angiography, in a low kV monoenergetic image, the heart and its valves as well as the coronary arteries may be segmented and labelled. The second processed data set may be a non-contrasted virtual non-iodine data set, in which an identification and quantification of calcium can be performed to yield a respective second intermediate result. In combination, by using the third sub-algorithm correctly quantified calcium values correctly assigned to coronary vessels and/or heart valves can be determined. In another example, in a contrasted low kV representation as first processed data set, a myocardium segmentation may be performed, while the second processed data set may be a functional representation, from which perfused blood volume information can be deduced using the second sub-algorithm. In combination, the third sub-algorithm may output perfused blood volume information for different segments or sections of the myocardium, for example standardized 18 sections, as quantitative evaluation result data. In concrete embodiments, for example, the segmented myocardium may be used as a mask by the third sub-algorithm of the evaluation algorithm. It is noted that perfused blood volume information may, alternatively, also be extracted from a iodine data set as second processed data set.

In another example, a tumor and an organ, for example the liver, may be segmented in at least one first processed data set, wherein the second intermediate result may, again, be a perfusion value, for example blood volume perfusion information. The result of the third sub-algorithm and of the evaluation algorithm as a whole may then be quantitative perfusion values of the tumor relative the organ.

Generally speaking, in such embodiments, the first sub-algorithm may be a segmentation algorithm as in principle known in the art. The second sub-algorithm may determine quantitative per-voxel results or a segmentation result regarding a material or generally, physical quantity, wherein the third sub-algorithm may combine the segmentations of the first and second intermediate result.

In an especially preferred embodiment, the first intermediate result comprises a segmented vessel tree, wherein the third sub-algorithm performs at least one fluid flow simulation in the segmented vessel tree to determine at least one fluid flow parameter as an evaluation result, wherein the simulation is at least partly parameterized using the second intermediate result. In particular, the segmented vessel tree may be a blood vessel tree, the fluid may be blood and the at least one fluid flow parameter may comprise a fractional flow reserve (FFR). In the state of the art, it has already been proposed to use segmented vessel trees for simulations, however, the quality of the simulation results may be reduced due to missing information. According to one or more example embodiments of the present invention, this missing information may be provided by the at least one second intermediate result, such that the first intermediate result provides the geometry of the vessel tree, in particular also a relevant organ like a heart, for example as a mesh, while further, in particular functional, information, that is simulation parameters, is provided in the second intermediate result. For example, in a iodine data set in angiography, the lumen of the vessels may be determined, while in a low kV monoenergetic image as second processed data set, a myocardium mass and other parameters may be determined. It should be noted that, in such an embodiment, a staged use of the principles discussed here may also be employed, such that, for example, a calcium image may also be determined as a processed data set, assisting in separating calcium from iodine and thus being able to better describe strongly calcified stenosis areas, which are difficult to segment, and/or include aneurysms into the simulation. In particular, other fluid flow parameters may also refer to such areas or aneurysms. In particularly preferred examples, additionally or alternatively, implants, for example stents, may be localized and/or precisely described based on high kV images, even allowing determination of fluid flow parameters, for example FFR, inside an implant, for example stent or stent graft.

In other preferred embodiments, at least a part of the first and at least a part of the second intermediate result may be used as quantitative input data to at least one disease value estimation of the third sub-algorithm. For example, the quantitative evaluation result data may comprise a disease score, in particular spatially resolved. Examples comprise calcium scores and a plaque classification. However, the disease value may also more concretely describe the disease, for example it may be determined whether a plaque is calcified, necrotic and/or to which share these classes apply. Such information may, for example, be deduced from monoenergetic data sets having as low energy as possible. In other embodiments, the disease value may also be a prediction value, for example quantify a percentage of a certain event occurring. In an example, from FFR values, risks for certain events and/or conditions may be quantitively calculated.

In advantageous embodiments, the third sub-algorithm additionally determines at least one two-dimensional output image visualizing the evaluation result data. Such output images may, in particular, also be derived from the image data set and/or at least one of the processed data sets. If a simulation is performed, of course, they may be completely newly generated. Such output images may provide a better understanding of the evaluation result data. In concrete embodiments, the orientation and/or view point and/or shown imaged region portion of the at least one output image based on the second processed data set and/or the second intermediate result is chosen based on the first intermediate result.

In concrete embodiments, for example, regarding calcification values, in particular calcification scores, the heart and valve orientation may be determined from the segmentation of the first processed data set and may define heart- and/or value-oriented views for displaying quantified calcium values in an output image. A heart-oriented view may, for example, comprise images are perpendicular to the short axis or the long axis of the heart. In particular, multiple output images along such axis may be provided. Of course, such heart-oriented views or valve-oriented views may also be used in other applications. For example, perfused blood volume information may be shown overlaid over the myocardium.

It is noted that another area of application of the principles described here is the assessment of implants. For example, if a stent or stent graft is implanted into a vessel, calcium may accumulate inside the stent. On the other hand, implants as strongly attenuating objects may be problematic in x-ray imaging. However, high kV representations, that is monoenergetic data sets, may clearly show the implant. This can be used to, for example, section-wise determine calcium or other occlusions inside the stent graft or stent, wherein, for example, the stent graft or stent may be removed from a processed data set while still using its position to evaluate materials inside the stent or stent graft. In particular, regarding output images, views into an implant, in particular a stent or stent graft, may be provided. If anatomical landmarks are determined from a low kV representation as first processed data set, such views into an implant may be oriented such that cross-sections of the vessel result as output images.

In embodiments of the present invention, at least one sub-algorithm may comprise a trained function. While, of course, also sub-algorithms using no artificial intelligence may be employed, the principles of the current invention are particularly advantageous if at least one of the sub-algorithms is a trained function. In general, a trained function mimics cognitive functions that humans associate with other human minds. In particular, by training based on training data the trained function is able to adapt to new circumstances and to detect and extrapolate patterns.

In general, parameters of a trained function can be adapted via training. In particular, supervised training, semi-supervised training, unsupervised training, reinforcement learning and/or active learning can be used. Furthermore, representation learning (an alternative term is “feature learning”) can be used. In particular, the parameters of the trained functions can be adapted intuitively by several steps of training.

In particular, a trained function can comprise a neural network, a support vector machine, a decision tree and/or a Bayesian network, and/or the trained function can be based on k-means Clustering, Q-learning, genetic algorithms and/or association rules. In particular, a neural network can be a deep neural network, a convolutional neural network or a convolutional deep neural network. Furthermore, a neural network can be an adversarial network, a deep adversarial network and/or a generative adversarial network.

One or more example embodiments of the present invention further concern an evaluation device for evaluating an image data set of an imaged region, wherein, from the image data set, different processed data sets having different image data content are determinable by image processing, the evaluation device comprising:

a first interface for receiving the image data set,

an image processing unit for determining at least two processed data sets having different image data content from the image data set,

an evaluation unit for determining quantitative evaluation result data describing at least one dynamic and/or static feature of the imaged region by applying an evaluation algorithm, and

a second interface for providing the evaluation result data,

wherein the evaluation unit comprises:

a first subunit for applying a first sub-algorithm of the evaluation algorithm to a first of the processed data sets to determine a first intermediate result relating to the image data content of the first processed image data set,

a second subunit for applying a second sub-algorithm of the evaluation algorithm to a second of the processed data sets to determine a second intermediate result relating to the image data content of the second processed data set, and

a third subunit for determining the quantitative evaluation data by a third sub-algorithm of the evaluation algorithm, wherein the third sub-algorithm uses both the first and the second intermediate results as input data.

In particular, the evaluation device is configured to perform a method according to one or more example embodiments of the present invention. All features and remarks regarding the method according to one or more example embodiments of the present invention analogously apply to the evaluation device according to one or more example embodiments of the present invention. Preferably, the evaluation device may comprise at least one processor and at least one storage device (or, alternatively, means or memory) and/or the functional units may be implemented at least partly by software and/or at least partly by hardware components.

An imaging device according to one or more example embodiments of the present invention has a control device comprising an evaluation device according to one or more example embodiments of the present invention. In particular, the imaging device may be a medical imaging device, for example a magnetic resonance device or, preferably, a computed tomography device. In particular regarding computed tomography, many acquisition techniques are known which result in image data sets, from which different processed data sets showing different image content can be determined. Regarding magnetic resonance devices, image data sets may be used which, for example in partial data sets, show different contrasts and/or from which processed data sets relating to different contrasts may be calculated. An exemplary embodiment from magnetic resonance imaging is the so-called Dixon technique, where partial data sets are acquired at different echo times to differentiate between different spin species, between which a chemical shift exists. For example, spins of protons bound in fat may be distinguished from spins of protons bound in water.

Providing the evaluation device as a part of an imaging device has the advantage of providing important information, in this case the high-quality quantitative evaluation result data, immediately where the image data set was acquired. However, it should be noted that the evaluation device may also be or be part of a viewing and/or evaluation work station, for example a PACS workstation (PACS—picture archiving and communication system).

A computer program according to one or more example embodiments of the present invention can be directly loaded into a storage device of an evaluation device and, if executed on the evaluation device, performs the steps of a method according to one or more example embodiments of the present invention. The computer program may be stored on an electronically readable storage medium according to one or more example embodiments of the present invention, which hence comprises control information comprising a computer program according to one or more example embodiments of the present invention, such that, when the electronically readable storage medium is used in an evaluation device, the evaluation device is configured to perform the steps of a method according to one or more example embodiments of the present invention. The electronically readable storage medium may be a non-transitory medium, for example a CD-ROM.

### BRIEF DESCRIPTION OF THE DRAWINGS

Other objects and features of the present invention will become apparent from the following detailed description considered in conjunction with the accompanying drawings. The drawings, however, are only principle sketches designed solely for the purpose of illustration and do not limit the present invention. The drawings show:

FIG. 1 a flowchart of a general embodiment of a method according to one or more example embodiments of the present invention,

FIG. 2 the functional structure of an evaluation device according to one or more example embodiments of the present invention, and

FIG. 3 a schematic view of an imaging device according to one or more example embodiments of the present invention.

### DETAILED DESCRIPTION

In the following, embodiments of the present invention are described with respect to a multi-energy computed tomography angiography data set as image data set to be evaluated. However, also other image data sets, from which processed data sets having different image content can be derived, can be used. The image data set of these embodiments has been acquired in detector-based multi energy computed tomography by using a counting x-ray detector. That is, all image data have been acquired in one single acquisition, such that derived processed data sets are all registered to each other.

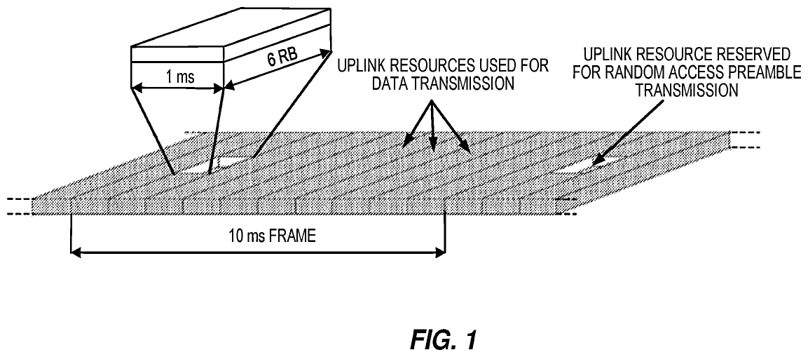

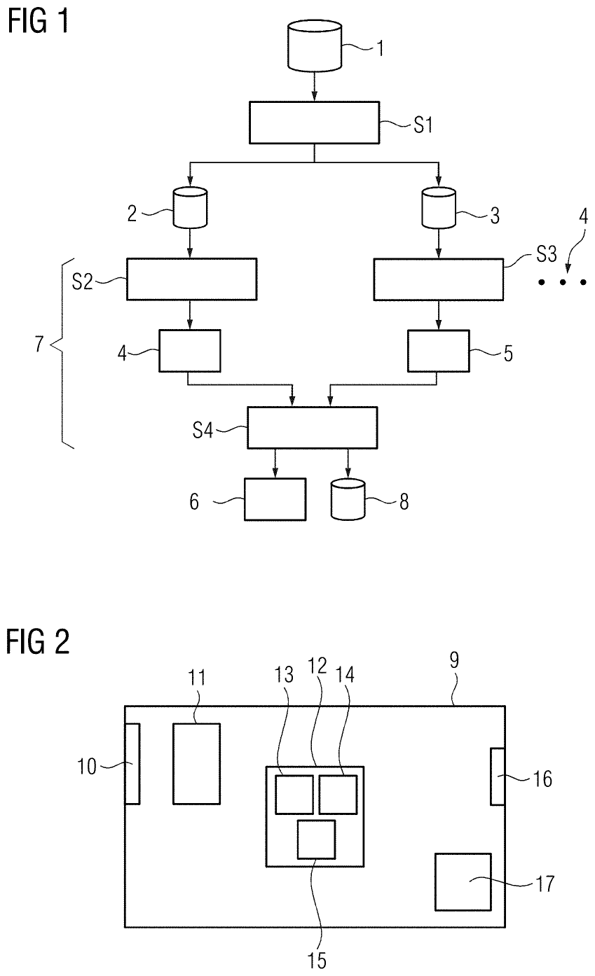

FIG. 1 shows a general flowchart of a method according to one or more example embodiments of the present invention. After the image data set one has been provided, in particular received via a first interface, in step S1, multiple processed data sets all having different image data content are derived from the image data set by image processing. For example, by using material decomposition, as principally known in the state of the art, virtual non-contrast images, iodine concentration images, functional images, virtual non-calcium images, virtual non-iodine images and the like can be determined. Additionally, or alternatively, monoenergetic images relating to certain energy intervals, for example defined energy slots or bins of the counting x-ray detector, may be determined. Each processed data set 2, 3 determined in this manner forms input data to at least one first or at least one second sub-algorithm of an evaluation algorithm configured to determine quantitative evaluation result data, for example calcium scores, FFR, perfused blood volume information and the like. In this embodiment, a first processed data set 2 is determined as input to a first sub-algorithm and a second processed data set 3 is determined as input data for a second sub-algorithm. However, this example is not limiting, and, as indicated by the dots 4, a larger number of first and/or second processed data sets 2, 3 and/or first and/or second sub-algorithms may be employed, in particular also in a stacked manner.

In a step S2, the first sub-algorithm is applied to the first processed data set 2, while in a step S3, the second sub-algorithm of the evaluation algorithm is applied to the second processed data set 3. In this manner, first and second intermediate results 4, 5 are determined. These intermediate results 4, 5 now both serve as input data for a third sub-algorithm of the evaluation algorithm, which is executed in step S4 to yield the quantitative evaluation result data 6. Additionally, the output data of the third sub-algorithm and hence the evaluation algorithm formed by the first sub-algorithm, the second sub-algorithm and the third sub-algorithm, and indicated as reference number 7 may also comprise at least one two-dimensional output image 8, in particular series of output images 8, visualizing or explaining the quantitative evaluation result data 6.

In this manner, from the same image data set 1, multiple processed data sets 2, 3, each showing different image data content, are derived, intermediate results 4, 5 are determined therefrom and together used as input data for a third sub-algorithm in step S4 to provide additional information and improved quality of the evaluation result data 6 and the output images 9. Processed data sets 2, 3 are already registered, such that no additional registration process is required. The different processed data sets can be understood as different representations of the image data set, each including different pieces of information which, put together, allow to provide an improved and reliable evaluation result.

In a first concrete embodiment, the first processed data set 2 may be a contrasted low-energy representation, from which, by the first sub-algorithm, as the first intermediate result 4, segmentation and labelling information regarding a vessel tree and/or organs in the imaged region of a patient is determined. In coronary angiography, the first intermediate result 4 thus describes the position and orientation of the heart and its valves as well as the lumen, course and labelling of the coronary arteries. As a second processed data set 3, a non-contrasted virtual non-iodine image is determined. Here, the second sub-algorithm provides, as second intermediate result 5, identification and quantification of calcium in the imaged region. The third sub-algorithm uses both intermediate results 4, 5 as input and determines correctly quantified calcium values correctly assigned to coronary vessels and/or heart valves. The output images 8 may be determined in a valve- and/or heart-oriented view, which view orientation and the corresponding positions may be determined from the first intermediate result. For example, a stack of two-dimensional MPR images may be determined along the long axis or the short axis of the heart.

In another embodiment or additionally, the or an additional second processed data set 3 may be a functional image, for example a late enhancement functional representation showing iodine, that is contrast agent, concentration in tissue, in particular the myocardium. From this functional representation, the second sub-algorithm may determine a perfused blood volume information as a second intermediate result 5, such that, after combination by the third sub-algorithm, for example perfused blood volume information may be shown overlaid over the myocardium in output images 8 in a heart-oriented view, while blood volume perfusion values may be quantitatively determined for different sections of the myocardium.

Furthermore, using a low energy monoenergetic image as the first processed data set 2 and a high-energy monoenergetic image as the second processed data set 3, anatomical landmarks and calcium information may be derived as first intermediate result 4 and the position and orientation of a stent or stent graft as intermediate result 5. Put together by the third sub-algorithm, a quantitative and visualized description of stent or stent graft occlusion by calcium can be derived in the manner of looking into the stent or stent graft.

In an especially advantageous concrete embodiment, the first processed data set 2 may be an iodine image, from which a segmented vessel tree is determined as first intermediate result 4. The second processed data set 2 is a low-energy monoenergetic image, from which further anatomical features may be segmented such that a set of parameters relevant for the simulation of fluid flow in the vessel tree, for example the myocardium mass, may be determined. The third sub-algorithm then performs a simulation of a blood flow in the vessel tree, which is parameterized using the second intermediate result 5. In this manner, at least one fluid flow parameter may be determined as evaluation result data, preferably at least the FFR (fractional flow reserve). In such embodiments, more than two processed data sets 2, 3 may be employed, for example by additionally using a virtual non-iodine image to detect calcifications and also take these into account in the simulation.

As a final concrete example, the first and second intermediate results 4, 5 may also be combined to determine at least one disease value using a respective disease value estimation in the third sub-algorithm. The disease value information may, in particular, comprise a trained function, such that, for example, a machine learning-based disease burden estimation is possible. In other examples, plaque quantification and classification may be performed.

But also generally, any of the first, second and third sub-algorithms may comprise trained functions, as already discussed above.

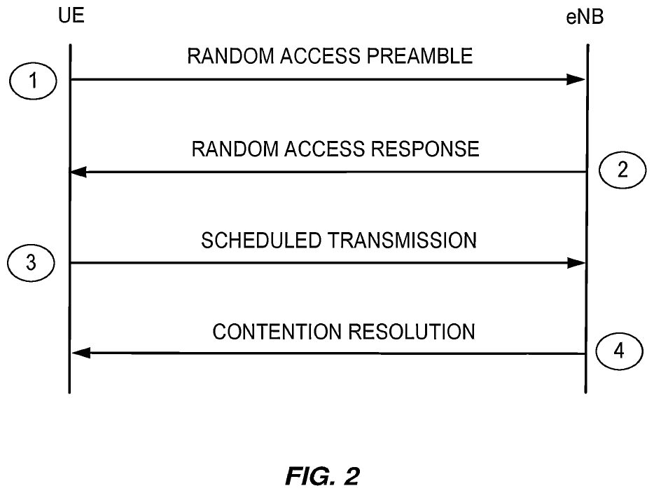

FIG. 2 shows the functional structure of an evaluation device 9 according to one or more example embodiments of the present invention. The evaluation device 9 comprises a first interface 10 for receiving the image data set 1. In an image processing unit 11, the processed data sets 2, 3 may be determined according to step S1. An evaluation unit 12 is provided for applying the evaluation algorithm 7 and comprises three subunits 13, 14 and 15, wherein the first subunit 13 executes the first sub-algorithm to determine the intermediate result 4, as described with regard to step S2, and the second subunit 14 executes the second sub-algorithm to determine the second intermediate result 5, as described with regard to step S3. Of course, as discussed above, multiple first and/or second subunits 13, 14 may also be provided and/or stacked structure of subunits 13, 14 and 15 may be present if the principles described herein are multiply used.

In the third subunit 15, the third sub-algorithm is performed to yield the quantitative evaluation result data 6 and optionally the at least one output image 8 described with respect to step S4. The evaluation result data 6 and the output images 8 may be provided via a second interface 16 of the evaluation device 9.

The evaluation device 9 may further comprise a storage device or memory 17 for storing data temporally or permanently for later retrieval, for example image data sets 1, processed data sets 2, 3, intermediate results 4, 5, evaluation result data 6 and output images 9.

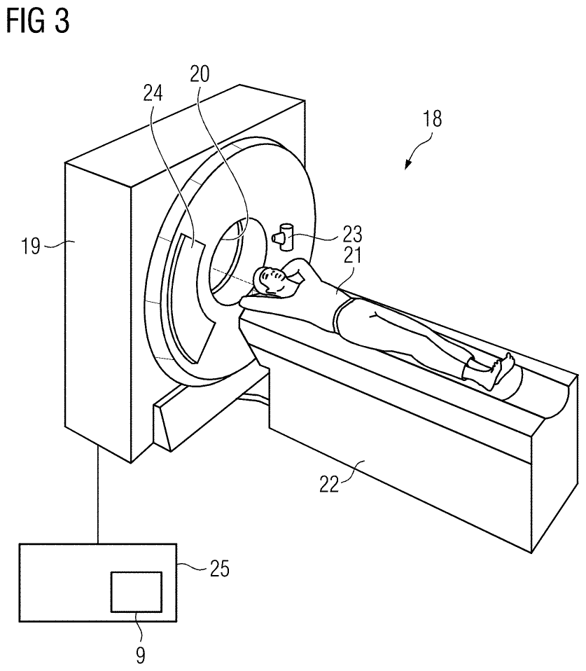

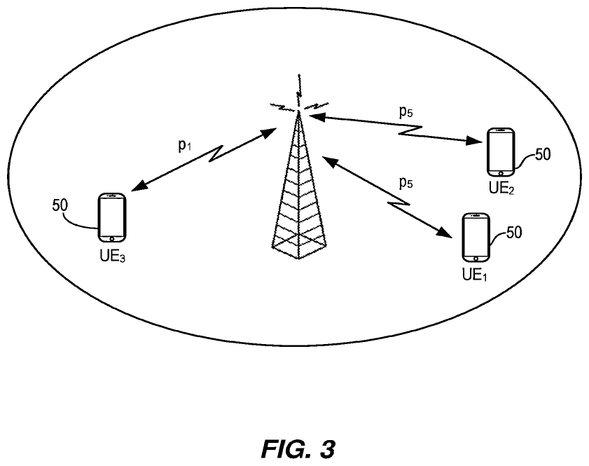

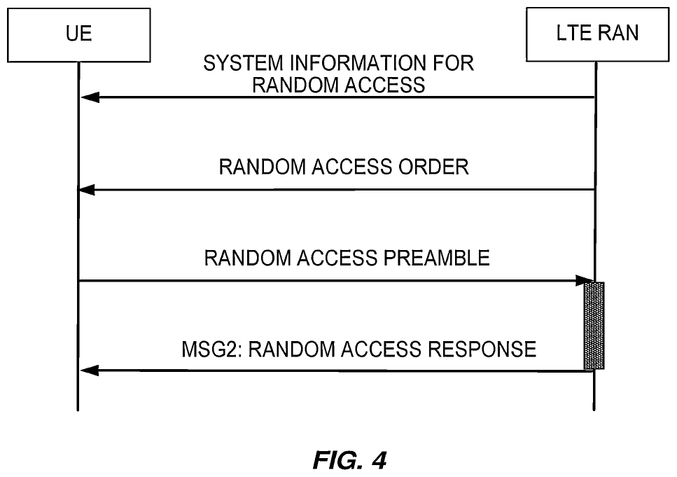

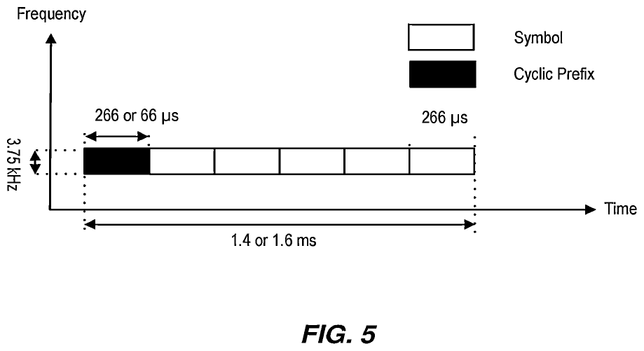

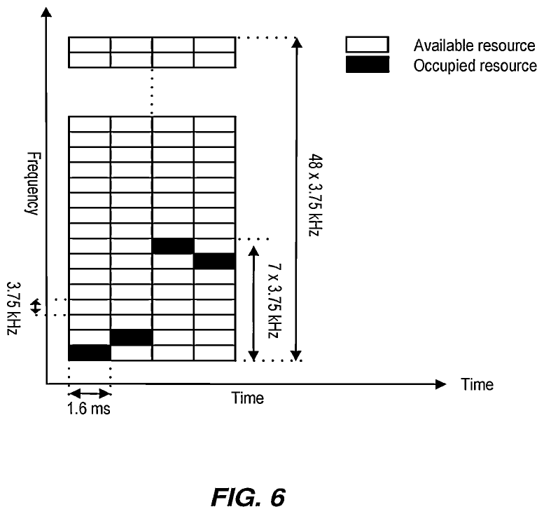

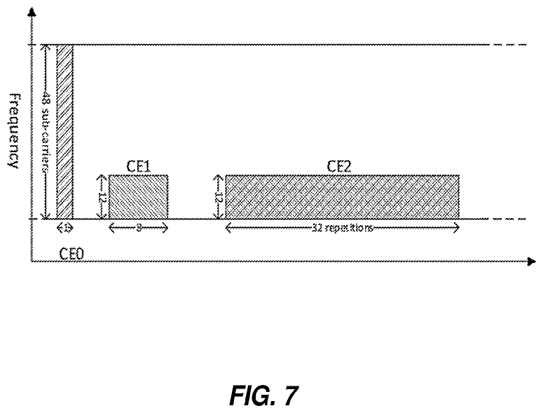

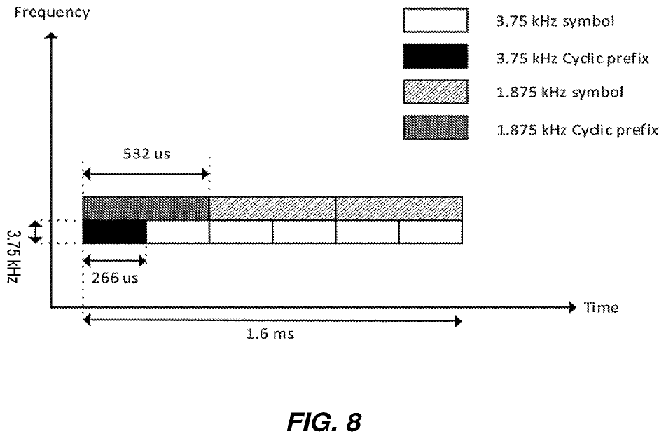

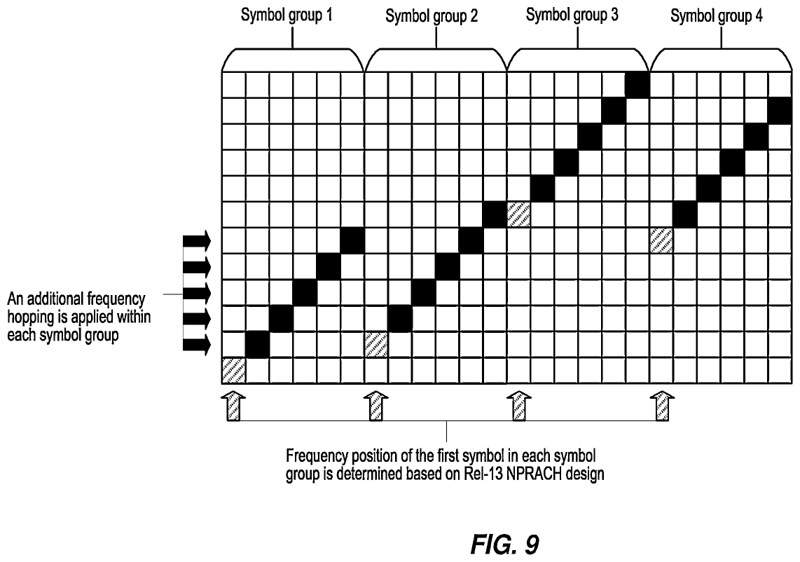

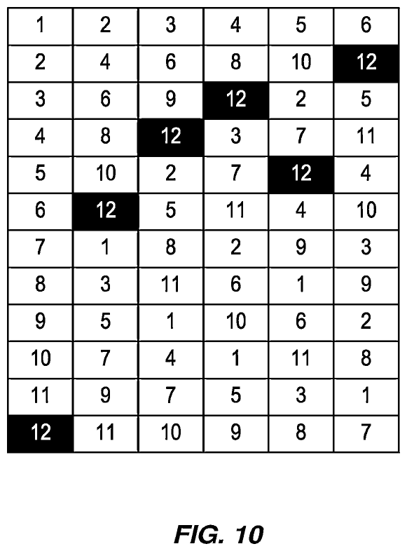

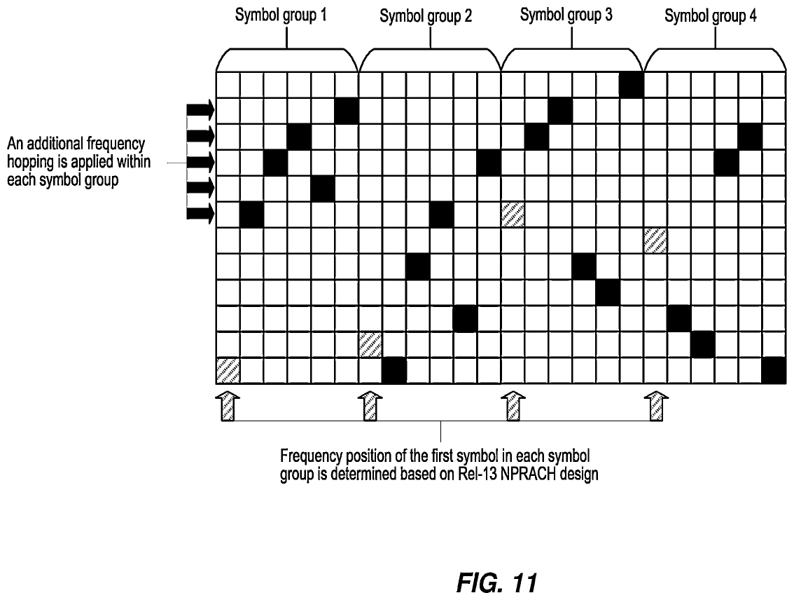

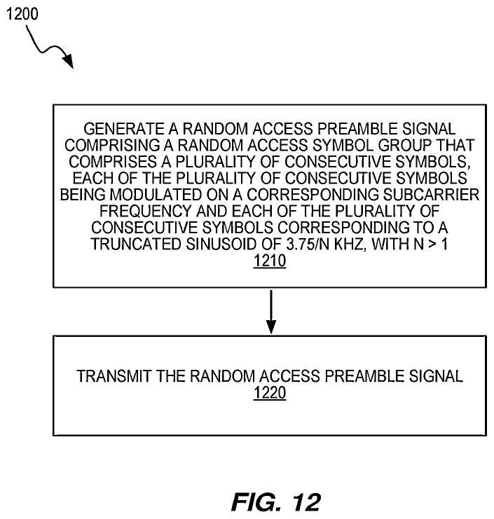

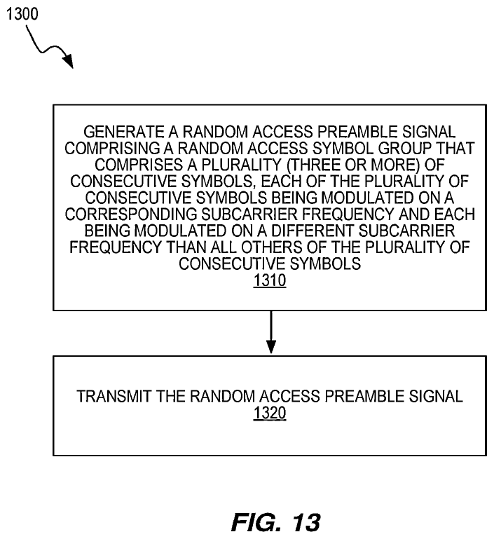

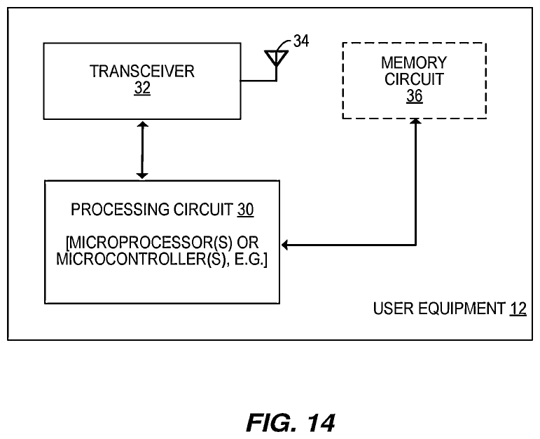

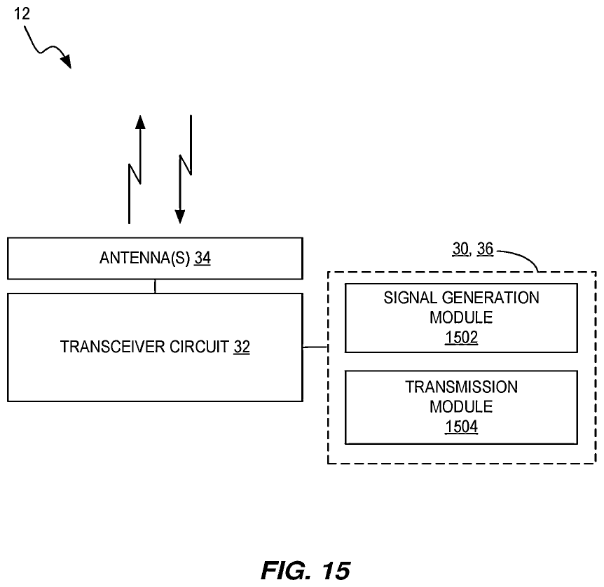

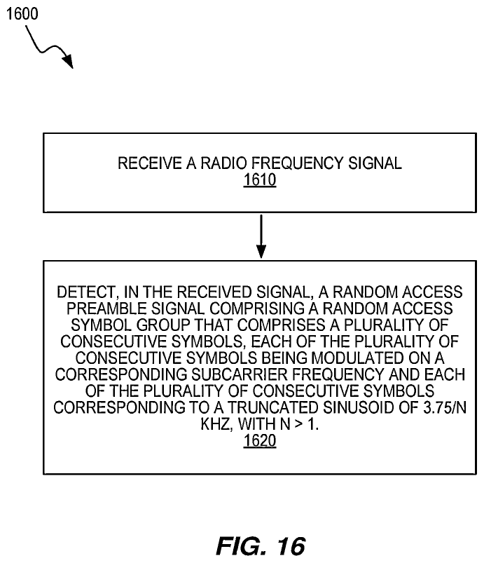

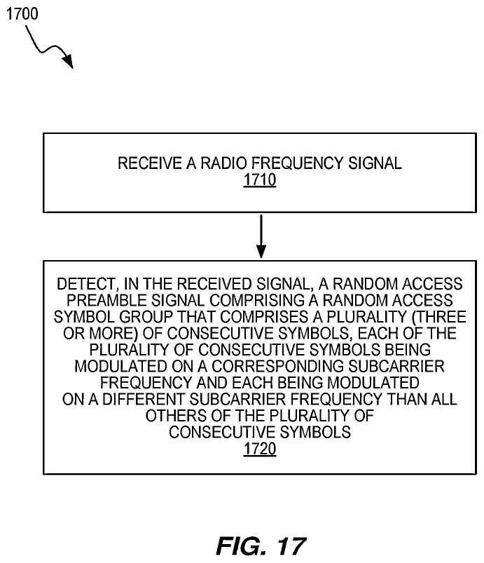

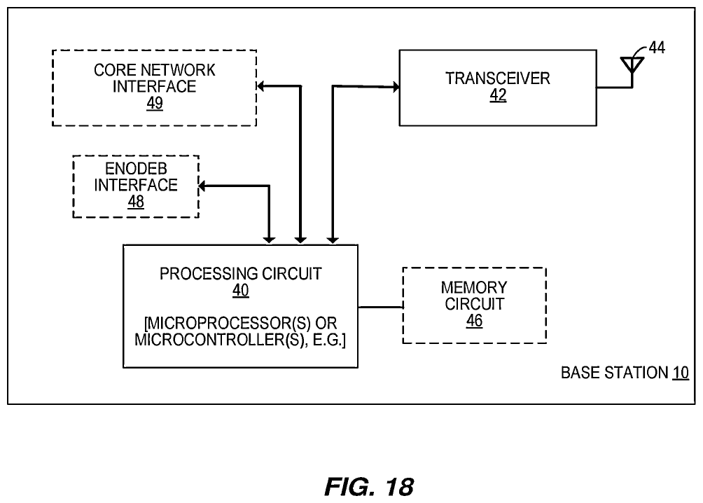

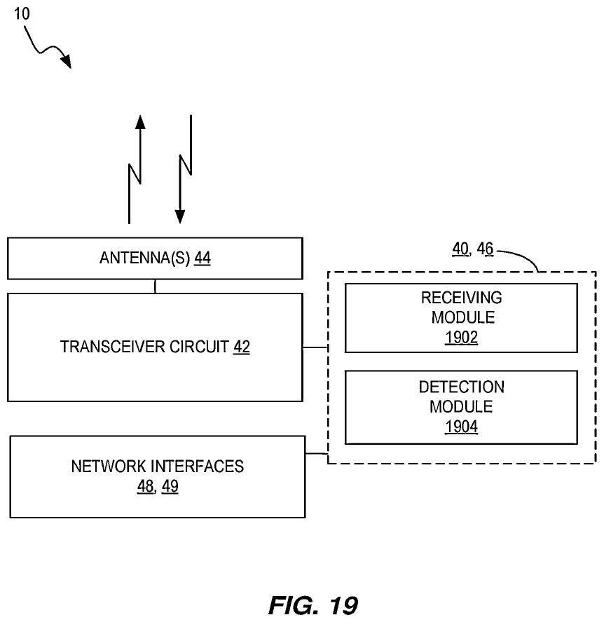

FIG. 3 schematically shows an imaging device 19 according to the current invention, in this case a computed tomography device. The imaging device 19 comprises a gantry 19 having a patient opening 20, into which a patient 21 may be introduced using a patient table 22. An acquisition assembly comprises an x-ray source 23 and an x-ray detector 24, in this case a counting x-ray detector 24, and may be rotated around the opening 20 and hence the patient to acquire projection images using different projection angles, from which an image data set 1 can be reconstructed.

The operation of the imaging device 18 is controlled by a control device 25, which, in this case, also comprises an evaluation device 9 according to one or more example embodiments of the present invention.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been described in detail with reference to the preferred embodiment, the present invention is not limited by the disclosed examples from which the skilled person is able to derive other variations without departing from the scope of the present invention.

What is claimed is:

1. A computer-implemented method for evaluating an image data set of an imaged region, wherein, from the image data set, different processed data sets having different image data content are determinable by image processing, and quantitative evaluation result data describing at least one of at least one dynamic feature or at least one static feature of the imaged region is determined by applying an evaluation algorithm, the method comprising:
determining, from the image data set, at least two processed data sets having different image data content;
applying a first sub-algorithm, of the evaluation algorithm, to a first of the at least two processed data sets to determine a first intermediate result relating to image data content of the first of the at least two processed data sets;
applying a second sub-algorithm, of the evaluation algorithm, to a second of the at least two processed data sets to determine a second intermediate result relating to image data content of the second of the at least two processed data sets; and
determining the quantitative evaluation result data by a third sub-algorithm of the evaluation algorithm, the third sub-algorithm using both the first intermediate result and the second intermediate result as input data.

2. The computer-implemented method according to claim 1, wherein
the image data set is a multi-energy computed tomography data set, and
at least one of the at least two processed data sets is determined at least one of based on a material decomposition or as a monoenergetic image.

3. The computer-implemented method according to claim 2, further comprising:
acquiring the image data set using at least one of a source-based or a detector-based multi-energy computed tomography.

4. The computer-implemented method according to claim 1, wherein the image data set is an angiography data set and at least one of the at least two processed data sets is selected from a virtual non-contrast image, an iodine concentration image, a functional image, a monoenergetic image, a virtual non-calcium image, or a virtual non-iodine image.

5. The computer-implemented method according to claim 1, wherein
the first intermediate result describes a segmentation result regarding multiple segmented features, and
the third sub-algorithm assigns data of the second intermediate result to segmented features to yield quantitative segmented feature-specific evaluation results.

6. The computer-implemented method according to claim 5, wherein the segmented features are anatomical features including at least one of (i) vessels or vessel segments of a vessel tree or (ii) organs or organ segments.

7. The computer-implemented method according to claim 1, wherein
the first intermediate result includes a segmented vessel tree,
the third sub-algorithm performs at least one fluid flow simulation in the segmented vessel tree to determine at least one fluid flow parameter as an evaluation result, and
the at least one fluid flow simulation is at least partly parametrized using the second intermediate result.

8. The computer-implemented method according to claim 7, wherein the segmented vessel tree is a blood vessel tree, the fluid is blood and the at least one fluid flow includes a fractional flow reserve.

9. The computer-implemented method according to claim 1, wherein at least a part of the first intermediate result and at least a part of the second intermediate result are used as quantitative input data to at least one disease value estimation of the third sub-algorithm.

10. The computer-implemented method according to claim 1, wherein the third sub-algorithm determines at least one two-dimensional output image visualizing the quantitative evaluation result data.

11. The computer-implemented method according to claim 10, wherein at least one of an orientation, a viewpoint, a shown imaged region portion of the at least one two-dimensional output image based on the second of the at least two processed data sets, or the second intermediate result is chosen based on the first intermediate result.

12. An evaluation device for evaluating an image data set of an imaged region, wherein, from the image data set, different processed data sets having different image data content are determinable by image processing, the evaluation device comprising:
a first interface to receive the image data set;
an image processor to determine, from the image data set, at least two processed data sets having different image data content;
an evaluation unit to determine quantitative evaluation result data describing at least one of at least one dynamic feature or at least one static feature of the imaged region by applying an evaluation algorithm;
a second interface to provide the quantitative evaluation result data; and
wherein the evaluation unit includes
a first sub-unit to apply a first sub-algorithm, of the evaluation algorithm, to a first of the at least two processed data sets to determine a first intermediate result relating to image data content of the first of the at least two processed data sets,
a second sub-unit to apply a second sub-algorithm, of the evaluation algorithm, to a second of the at least two processed data sets to determine a second intermediate result relating to image data content of the second of the at least two processed data sets, and
a third sub-unit to determine the quantitative evaluation result data by a third sub-algorithm of the evaluation algorithm, the third sub-algorithm using both the first intermediate result and the second intermediate result as input data.

13. An imaging device with a control device comprising:
the evaluation device according to claim 12.

14. A non-transitory computer-readable storage medium storing a computer program that, when executed by at least one processor at an evaluation device, causes the evaluation device to perform the method of claim 1.

15. An evaluation device to evaluate an image data set of an imaged region, the evaluation device comprising:
a memory storing computer-executable instructions; and
at least one processor configured to execute the computer-executable instructions to cause the evaluation device to
determine, from the image data set, at least two processed data sets having different image data content,
apply a first sub-algorithm, of an evaluation algorithm, to a first of the at least two processed data sets to determine a first intermediate result relating to image data content of the first of the at least two processed data sets,
apply a second sub-algorithm, of the evaluation algorithm, to a second of the at least two processed data sets to determine a second intermediate result relating to image data content of the second of the at least two processed data sets, and
determine quantitative evaluation result data by a third sub-algorithm of the evaluation algorithm, the third sub-algorithm using both the first intermediate result and the second intermediate result as input data, and the quantitative evaluation result data describing at least one of at least one dynamic feature or at least one static feature of the imaged region.

16. The computer-implemented method according to claim 3, wherein the acquiring acquires the image data set using a counting x-ray detector.

17. The computer-implemented method according to claim 4, wherein the functional image is a perfusion image.

18. The computer-implemented method according to claim 5, wherein
the first intermediate result includes a segmented vessel tree,
the third sub-algorithm performs at least one fluid flow simulation in the segmented vessel tree to determine at least one fluid flow parameter as an evaluation result, and
the at least one fluid flow simulation is at least partly parametrized using the second intermediate result.

19. The computer-implemented method according to claim 18, wherein the segmented vessel tree is a blood vessel tree, the fluid is blood and the at least one fluid flow includes a fractional flow reserve.

20. The computer-implemented method according to claim 5, wherein at least a part of the first intermediate result and at least a part of the second intermediate result are used as quantitative input data to at least one disease value estimation of the third sub-algorithm.
